(19) United States
(12) Patent Application Publication
(10) Pub. No.: US 2025/0127450 A1
(43) Pub. Date: Apr. 24, 2025

(54) PEDIATRIC DETECTION AND ALL AGE GROUPS CLASSIFICATION FROM ECG

(71) Applicant: KONINKLIJKE PHILIPS N.V., Eindoven (NL)

(72) Inventors: Junmo An, Cambridge, MA (US); Richard Earl Gregg, Cambridge, MA (US)

(21) Appl. No.: 18/915,543

(22) Filed: Oct. 15, 2024

Related U.S. Application Data
(60) Provisional application No. 63/544,832, filed on Oct. 19, 2023.

(51) Int. Cl. A61B 5/346; A61B 5/00

(52) U.S. Cl. CPC A61B 5/346; A61B 5/7267; A61B 5/742

(57) ABSTRACT

A method for analysis of an ECG input for a subject to determine whether the subject is a pediatric subject or non-pediatric subject, and to classify the subject in one of a plurality of different age groups, comprising: receiving an ECG input for the subject; determining that the subject is a pediatric or non-pediatric subject; upon determining that the subject is a pediatric subject, determining that the subject belongs in one of a plurality of different pediatric age groups; upon determining that the subject is a non-pediatric subject, determining that the subject belongs in one of a plurality of different non-pediatric age groups; and performing an automated analysis of the received ECG input for the subject, wherein the automated analysis is based in part on the determined age group in which the subject belongs.

### FIELD OF THE DISCLOSURE

The disclosed subject matter generally pertains to methods for pediatric detection and all age groups classification from ECG.

### BACKGROUND

Electrocardiogram (ECG) is a non-invasive test that measures the electrical activity of the heart by placing electrodes on the chest and limbs. It is a vital diagnostic tool in cardiology, used to identify a wide range of cardiac conditions, including heart attack, arrhythmia, and heart failure. ECG can also be used to monitor the effectiveness of treatment and detect early signs of complications.

Accurate knowledge of the patient's age is essential for selecting ECG criteria that are appropriate, which has a significant impact on the accuracy of diagnoses in all age groups. Accurate and timely interpretation of multi-lead (e.g., 3-, 4-, 5-, 12-, 15-, 16-, 22-, 80-lead, etc.) ECGs is critical for diagnosing cardiac arrhythmias and abnormalities, whether performed by clinicians, automated ECG analysis software, or ECG machines (electrocardiogramawever, automated ECG analysis frequently omits age information, presenting a challenge in both pediatric and adult cases. Also, it may be difficult for clinicians to identify the patient's age, especially in urgent scenarios like emergency rooms and ambulances. Pediatric and adult ECGs can vary significantly, and age is a critical factor in determining accurate ECG thresholds. This can significantly impact the accuracy of diagnoses in patients of all ages. Therefore, misclassifying ECGs without age information poses a significant risk of misdiagnosing cardiac diseases.

Automated ECG analysis software often omits age information, and clinicians may not be able to identify a patient's age in emergency situations. If age information is unknown, clinicians and ECG analysis software may not be able to diagnose accurately, as ECG criteria for accurate diagnosis vary significantly by age, especially in pediatrics.

### SUMMARY OF THE DISCLOSURE

There it thus a continued need to determine the age of a subject for which ECG data is received and analyzed, to ensure accurate diagnosis. This results in significant improvements in diagnosis and care of a subject.

Various embodiments and implementations are directed to methods and systems for pediatric detection and all age groups classification from ECG. A system receives an ECG input for a subject and analyzes the input to determine whether the subject is a pediatric or non-pediatric subject. If the subject is determined to be a pediatric subject, then the subject is classified by the system into one of a plurality of different pediatric age groups. If the subject is determined to be a non-pediatric subject, then the subject is classified by the system into one of a plurality of different non-pediatric age groups.

According to an aspect, a method for analysis of an ECG input for a subject to determine whether the subject is a pediatric subject or non-pediatric subject, and to classify the subject in one of a plurality of different age groups based on an estimated age of the subject is provided. The method includes: receiving an ECG input for the subject, wherein the ECG input does not comprise or is not accompanied with an age for the subject; determining, by a trained algorithm based on the received ECG input, that the subject is a pediatric or non-pediatric subject; upon determining that the subject is a pediatric subject, determining, by the trained algorithm based on the received ECG input, that the subject belongs in one of a plurality of different pediatric age groups; upon determining that the subject is a non-pediatric subject, determining by the trained algorithm based on the received ECG input, that the subject belongs in one of a plurality of different non-pediatric age groups; and performing, using an automated ECG analysis tool, an automated analysis of the received ECG input for the subject, wherein the automated analysis is based in part on the one of a plurality of different pediatric age groups or the one of a plurality of different non-pediatric age groups in which the subject belongs.

According to an embodiment, the method further includes extracting one or more features from the received ECG input, wherein the extracted one or more features are provided as input to the trained algorithm.

According to an embodiment, the trained algorithm is a trained machine learning algorithm or an end-to-end deep learning algorithm.

According to an embodiment, the algorithm is trained using multi-lead ECG data for a plurality of subjects at a plurality of different ages.

According to an embodiment, a plurality of features are extracted from the multi-lead ECG data for each of the plurality of subjects, and wherein the plurality of extracted features are used to train the algorithm.

According to an embodiment, the plurality of extracted features are reduced via dimensionality reduction prior to training the algorithm.

According to an embodiment, the method further includes displaying, via a user interface, the determined one of a plurality of different non-pediatric age groups or determined one of a plurality of different non-pediatric age groups in which the subject belongs.

According to an embodiment, the method further includes displaying, results of the automated analysis.

According to an embodiment, the method further includes receiving, from a clinician via a user interface, a selection of a preferred age range, wherein the determined one of a plurality of different non-pediatric age groups or determined one of a plurality of different non-pediatric age groups in which the subject belongs comprises the preferred age range.

According to another aspect is a system for analysis of an ECG input for a subject to determine whether the subject is a pediatric subject or non-pediatric subject, and to classify the subject in one of a plurality of different age groups based on an estimated age of the subject. The system includes: ECG input for the subject, wherein the ECG input does not comprise or is not accompanied with an age for the subject; a trained algorithm, wherein the trained algorithm is trained to determine whether the subject is a pediatric subject or non-pediatric subject, and to classify the subject in one of a plurality of different age groups based on an estimated age of the subject; a processor configured to: (i) determine, by the trained algorithm based on the ECG input, that the subject is a pediatric or non-pediatric subject; (ii) upon determining that the subject is a pediatric subject, determine, by the trained algorithm based on the ECG input, that the subject belongs in one of a plurality of different pediatric age groups or upon determining that the subject is a non-pediatric subject, determine by the trained algorithm based on the ECG input, that the subject belongs in one of a plurality of different non-pediatric age groups; and (iii) perform, using an automated ECG analysis tool, an automated analysis of the received ECG input for the subject, wherein the automated analysis is based in part on the one of a plurality of different pediatric age groups or the one of a plurality of different non-pediatric age groups in which the subject belongs.

According to an embodiment, the processor is further configured to extract one or more features from the ECG input, wherein the extracted one or more features are provided as input to the trained algorithm.

According to an embodiment, the processor is further configured to cause the display, via a user interface, of the determined one of a plurality of different non-pediatric age groups or determined one of a plurality of different non-pediatric age groups in which the subject belongs.

According to an embodiment, the processor is further configured to cause the display, via a user interface, of results of the automated analysis.

According to an embodiment, the processor is further configured to receive, from a clinician via a user interface, a selection of a preferred age range, wherein the determined one of a plurality of different non-pediatric age groups or determined one of a plurality of different non-pediatric age groups in which the subject belongs comprises the preferred age range.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 illustrates examples of pediatric and adult 12-lead ECGs.

FIG. 2 illustrates a flowchart of the system for pediatric detection and all age groups classification, in accordance with an embodiment.

FIG. 3 illustrates a flowchart of the procedure of the data preparation to train, text, and deploy the machine learning (ML) model for classification or regression, in accordance with an embodiment.

FIG. 4 illustrates a flowchart of the procedure of the data preparation to train, text, and deploy the end-to-end deep learning (DL) model for classification or regression, in accordance with an embodiment.

FIG. 5 illustrates examples of 10-second 12-lead ECG (RawECG) and corresponding representative average beat (RepBeat), in accordance with an embodiment.

FIG. 6 illustrates a flowchart to train, text, and deploy the feature-based machine learning (ML) and end-to-end deep learning (DL) models, in accordance with an embodiment.

FIG. 7 illustrates examples of ECG signal and corresponding interpretations for pediatric cases, in accordance with an embodiment.

FIG. 8 is a schematic representation of an ECG analysis system, in accordance with an embodiment.

### DETAILED DESCRIPTION OF EMBODIMENTS

As noted above, ECG criteria for diagnosis can vary significantly by age and without age information, clinicians and ECG analysis software may have difficulty making accurate diagnoses. Described or otherwise envisioned herein are methods and systems for pediatric detection and all age groups classification from ECG, which can benefit clinicians, automated ECG analysis software, and ECG machines (electrocardiogramproving the accuracy of cardiac abnormality and arrhythmia diagnosis. This can lead to early detection and treatment of heart disease, improved patient outcomes, and reduced risk of complications.

Electrocardiogram (ECG) is a non-invasive test that measures the electrical activity of the heart by placing electrodes on the chest and limbs. It is a vital diagnostic tool in cardiology, used to identify a wide range of cardiac conditions, including heart attack, arrhythmia, and heart failure. ECG can also be used to monitor the effectiveness of treatment and detect early signs of complications.

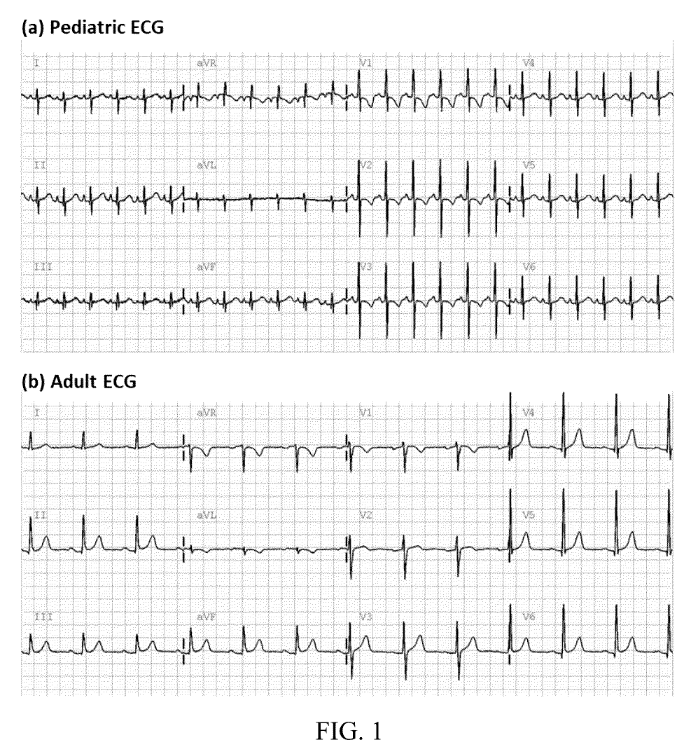

Pediatric ECG criteria for diagnosis can vary greatly with age, particularly in the early years. Accurate knowledge of the patient's age is essential for selecting ECG criteria that are appropriate, which has a significant impact on the accuracy of diagnoses in all age groups. Misclassifying ECGs without age information poses a significant risk of misdiagnosing cardiac diseases. FIG. 1 shows examples of pediatric (a) and adult (b) 12-lead (Lead I, II, III, aVR, aVL, aVF, V1, V2, V3, V4, V5, and V6) ECGs.

The innovative approach described herein, encompassing pediatric detection and all age classification (i.e., different pediatric and adult groups) using ECG data, provides valuable solutions for clinicians and automated ECG interpretation/analysis machines. It enhances the accuracy of diagnosing and interpreting cardiac abnormalities and arrhythmias across all age groups, benefiting clinicians, automated ECG analysis software, and electrocardiogramachines. The innovative approach described herein develops feature-based machine learning models and end-to-end deep learning models that can identify pediatric patients from adults and classify them into different pediatric age groups (neonates, infants, children, adolescents, and different adult age groups) using 12-lead ECGs. It has previously been impossible using an automated approach to identify pediatric patients from adults and classify them into different pediatric age groups (neonates, infants, children, adolescents) and different adult age groups using 12-lead ECGs.

Key components include feature-based machine learning (ML) models and end-to-end deep learning (DL) models that can identify pediatric patients from adults, classify them into different pediatric age groups (neonates, infants, children, adolescents, and adults) from 12-lead ECG data. AI explainability has enabled the discovery of a new feature for feature-based ML. This invention significantly improves the accuracy of cardiac abnormality and arrhythmia diagnosis for clinicians, automated ECG analysis software, and ECG machines.

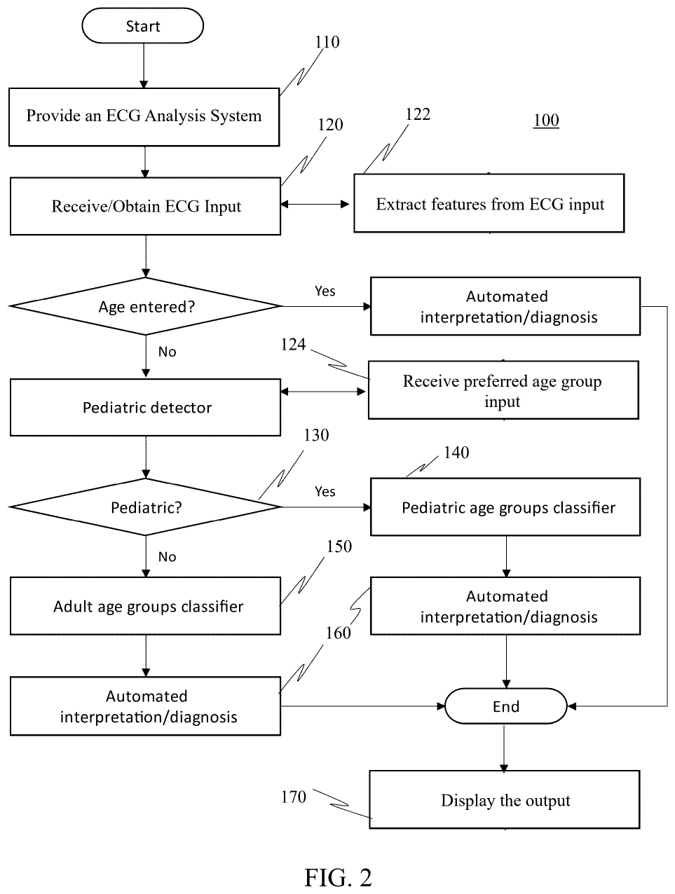

FIG. 2 shows a flowchart of a system for pediatric detection and all age groups (i.e., different pediatric and adult groups) classification from multi-lead ECGs, in accordance with an embodiment. If clinicians do not enter or miss the patient's age in automated ECG analysis software (e.g., Philips DXL ECG algorithm), pediatric detection and all age group classification algorithms based on feature-based ML or end-to-end DL models can be used to infer the patient's age from their ECG.

Types of multi-lead ECG are as below:
12-lead ECG (default)
12-lead additions
a. Left-sided Posterior (V7L, V8L, V9L)
b. Right-sided Posterior (V7R, V8R, V9R)
c. Right-sided Anterior (V3R, V4R, V5R, V6R)
d. 22-lead ECG (V3R, V4R, V5R, V6R, V7L, V8L, V9L) for default mode only (not for self mode)
e. 80-lead ECG (58 anterior, 12 lateral, and 10 posterior lead data) for default mode only (not for self mode)
5-lead ECG
4-lead ECG
3-lead ECG
2-lead ECG

Because the age ranges of pediatric and adult age groups vary, users can select one of the preferred range options. Selective options of different pediatric and adult age groups can be classified as follows. Note that the estimated age by this invented system can also be displayed if the user selects it from the menu. For pediatric cases, the age will be presented in months if the child is younger than one year old, and in years otherwise. For adult ages, the age will always be displayed in years.

### Pediatric (Option 1):

a. Neonates (0 day to 1 month)
b. Infants (1 month to 2 years)
c. Children (2 to 12 years)
d. Adolescents (12 to 16 or 17 years)

### Pediatric (Option 2):

a. Newborn (0 day to 1 month)
b. Infant (1 month to 1 year)
c. Toddler (1 to 3 years)
d. Preschool (3 to 6 years)
e. School age child (6 to 12 years)
f. Adolescent (12 to 18 years)

### Adult (Option 1):

a. Young adults (17 or 18 to 35 years)
b. Middle-aged adults (35 to 65 years)
c. Older adults (65+ years)

### Adult (Option 2):

a. Young or early adulthood (approximately aged 20-39)
b. Middle adulthood (40-59)
c. Young old (60-75) and old old (75 and up) (or old age (60+))

### Adult (Option 3):

a. Young adult (18-25 age)
b. Adult (26-44 age)
c. Middle-age (45-59 age)
d. Old age (60 age)

FIG. 2 will be discussed in greater detail hereinafter. Pediatric detection and classification across all age groups (i.e., different pediatric and adult groups) can be accomplished using either feature-based machine learning (ML) or end-to-end deep learning (DL) approaches. Feature-based ML is a type of ML where a model is trained on a set of features. Features are characteristics of the data that are used to make predictions. End-to-end DL is a type of ML where a model is trained to learn features from the data itself. This means that the model does not need to be explicitly told which features are important. Instead, the model learns to identify the features that are most predictive of the target variable. Feature-based ML learning models are typically less complex than end-to-end DL models. Feature-based ML learning models can be trained on smaller datasets than end-to-end DL models. Feature-based ML learning models are typically more interpretable than end-to-end DL models. End-to-end DL models can achieve state-of-the-art results on many tasks, but can be computationally expensive to train and require a large amount of data.

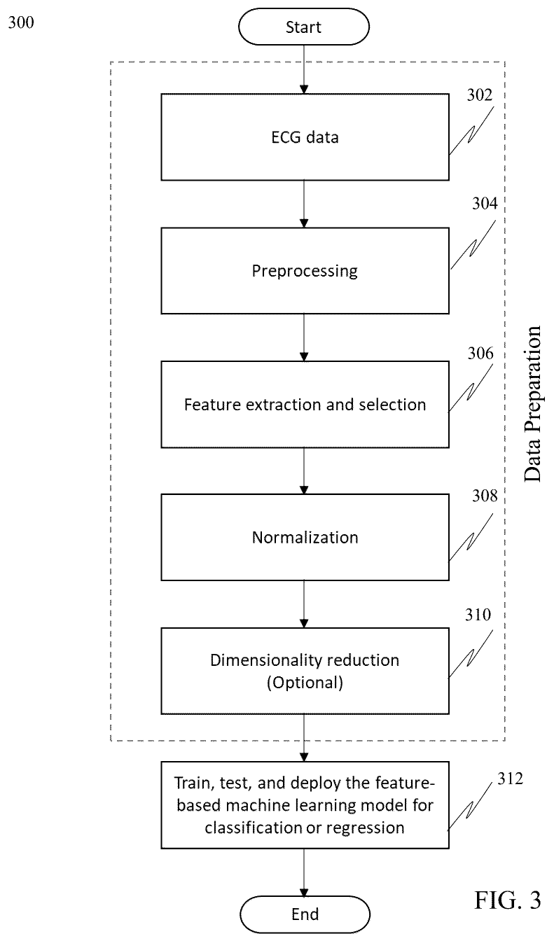

FIG. 3, in accordance with an embodiment, shows a flowchart of the procedure of the data preparation to train, test, and deploy the ML model for classification or regression. For feature-based ML, various features can be extracted and chosen from multi-lead ECG data, either through automated selection or expert-driven manual selection. These features encompass morphological attributes, heart rate variability (HRV) characteristics, frequency parameters, and statistical metrics, and they are derived from elements such as heart rate, P-wave, PR interval, QRS complex, Q-wave, QT interval, T-wave, among others. To automatically identify the most significant features, methods like permutation importance, tree-based techniques (e.g., decision trees, random forests, gradient boosting machines, etc.), Lasso and Ridge regression, information gain, correlation analysis, recursive feature elimination (RFE), partial least squares (PLS), Shapley values, and others can be applied. More detailed descriptions of morphological features, HRV features, frequency features, and statistical features are provided below.

Possible morphological features: Amplitude, duration, slope, area under the curve, peak-to-peak amplitude, asymmetry, skewness, kurtosis, rise time, fall time, overshoot, undershoot, baseline wander, frequency, phase, and others.

Possible HRV features: Maximum HRV, minimum HRV, mean HRV, median HRV, variance of HRV, standard deviation of HRV, pNN20, pNN50, standard deviation of successive differences (SDSD), standard deviation of normal-to-normal intervals (SDNN), root mean square of successive differences (RMSSD), triangular index (TINN), heart rate turbulence (HRT), percentage of successive differences greater than 5 milliseconds (pNN5), percentage of successive differences greater than 10 milliseconds (pNN10), low frequency (LF) power, high frequency (HF) power, LF/HF ratio, total power, and others.

Possible frequency features using FFT (Fast Fourier Transform): Mean, variance, entropy, energy, skew, kurtosis, power spectral density (PSD), peak frequency, bandwidth, spectral entropy, spectral kurtosis, magnitude spectrum mean, magnitude spectrum median, magnitude spectrum mode, magnitude spectrum standard deviation, magnitude spectrum skewness, phase spectrum mean, phase spectrum median, phase spectrum mode, phase spectrum standard deviation, phase spectrum skewness, coherence spectrum magnitude, and coherence spectrum phase.

Possible types of FFT methods: Welch method, Multitaper method, Pisarenko method, Maximum entropy method (MEM), Burg method (Autoregressive power spectral density estimate), Multiple signal classification (MUSIC) method, Bartlett method, Blackman-Tukey method, Thomson method, Capon method, Yule-Walker method, Minimum variance distortionless response (MVDR) method, and others.

Possible statistical features: Maximum, minimum, mean, median, variance, standard deviation, skewness, kurtosis, Shannon entropy, Tsallis entropy, Rényi entropy, percentile features (e.g., P10, P25, P50, P75, P90), interquartile range, range, coefficient of variation, decile features (e.g., D1, D2, . . . , D9, D10), moments of the distribution (e.g., first moment, second moment, third moment, . . . ), central tendency measures (e.g., mode, median, mean), dispersion measures (e.g., variance, standard deviation, interquartile range), shape measures (e.g., skewness, kurtosis), and others.

To reduce the number of features in the extracted data from ECG, we can optionally use integrated linear and nonlinear dimensionality reduction algorithms, also known as manifold learning. This can be done using a variety of methods, such as principal component analysis (PCA), multidimensional scaling (MDS), locally linear embedding (LLE), isomap embedding, t-distributed stochastic neighbor embedding (t-SNE), diffusion maps, kernel PCA, factor analysis, graph embedding, spectral clustering, autoencoders, and others. These methods may help to improve the detection of pediatric and the classification of all age groups.

The features extracted from ECG data can be utilized as input for training well-established machine learning classifiers, including but not limited to the multi-layer perceptron network (MLP), k-nearest neighbors (kNN), radial basis function kernel support vector machines (RBF SVM), Gaussian Naive Bayes, Gaussian process, decision tree, random forest, stochastic gradient descent (SGD), quadratic discriminant analysis (QDA), logistic regression, support vector machine (SVM), AdaBoost, Gradient boosting machines (GBMs), XGBoost, LightGBM, CatBoost, Neural networks, and various other classifiers.

Flowchart 300 begins with data preparation at step 302 with the ECG data. In accordance with the process described above, preprocessing is performed at step 304. At step 306 features are extracted and selected. At step 308 normalization is performed. At optional step 310 dimensionality reduction is performed. Following the data preparation, step 312 trains, tests, and deploys the feature-based machine learning model for classification or regression.

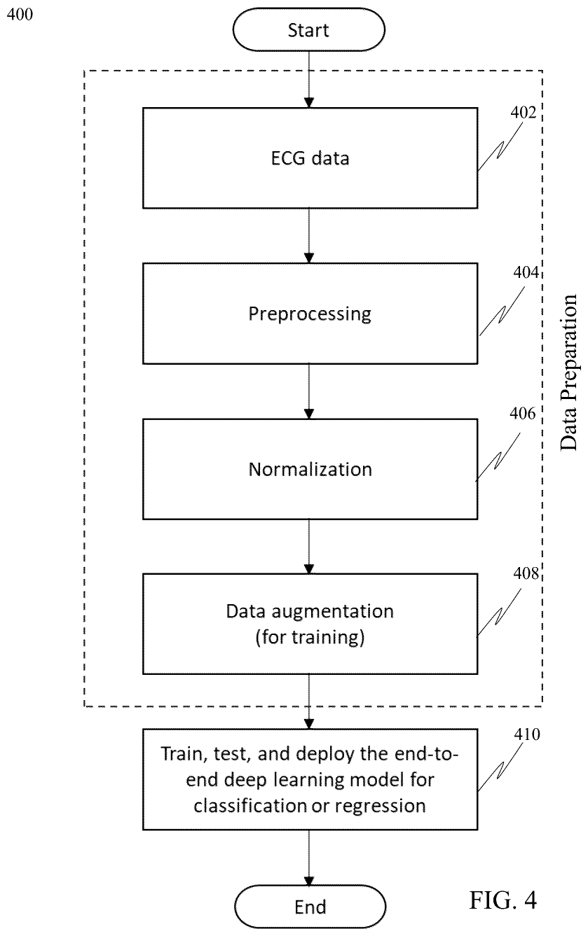

FIG. 4, in accordance with an embodiment, shows the flowchart of the procedure of the data preparation to train, test, and deploy the end-to-end deep learning (DL) model for classification or regression. Two different types of input data can be typically used for an end-to-end DL model: raw 10-second 12-lead ECG (RawECG) and the average representative beat (RepBeat).

Flowchart 400 begins with data preparation at step 402 with the ECG data. In accordance with the process described above, preprocessing is performed at step 404. At step 406 normalization is performed. At step 408 data augmentation (for training) is performed. Following the data preparation, step 410 trains, tests, and deploys the end-to-end deep learning model for classification or regression.

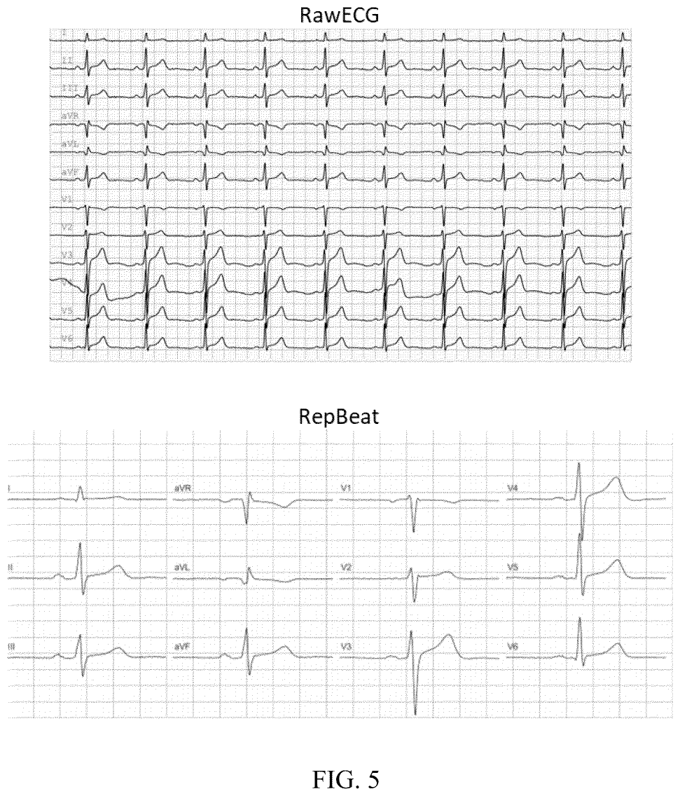

FIG. 5, in accordance with an embodiment, shows the examples of RawECG and corresponding RepBeat. Data has showed that using raw ECG data as input to a deep neural network (DNN) outperformed using RepBeat input for pediatric detection and age group classification. However, training time using RepBeat input was much faster. Therefore, RepBeat input could be integrated with selected HRV features on a dense layer (a.k.a., a fully connected layer) of a DNN and then applied to the output layer activation function (sigmoid for pediatric detection and softmax for all age group classification). The procedure for extracting RepBeat from a 12-lead ECG involves several steps:
1. Extract and align the ECG complexes from all 12 leads.
2. For each sample point in the aligned ECG complexes, select a value, such as the mean or median, after removing outliers.
3. String the sample points together to generate a representative ECG complex, or RepBeat.
4. Optionally, perform additional signal processing on the RepBeat, such as averaging over 1200 milliseconds and selecting a segment (e.g., 800 milliseconds) centered on the QRS complex.

For end-to-end DL model, deep neural network architectures such as long short-term memory (LSTM) networks, convolutional neural networks (CNNs) such as AlexNet, SqueezeNet, GoogLeNet, ShuffleNet, MobileNet, VGG, ResNet, SE-ResNet, ResNeXt, DenseNet, SENet, Inception, NASNet, Transformers, Vision Transformers (ViTs), EfficientNet, DarkNet, YOLO, Mask R-CNN, and pre-trained models can be used for pediatric detection and all age groups classification. During the training phase of the DL model, data augmentation techniques such as amplifying, band-pass filter, baseline shift, baseline wander addition, Cabrera lead sequence, chest leads shuffling, cutout, dropout, electromyographic (EMG) noise, Flow-Mixup, Gaussian blur, Gaussian noise addition, high-pass filter, horizonal flip, lead removal, leads order reversal, leads order shuffling, line noise addition/removal, low-pass filter, Mixup, scaling, sigmoid compression, time-window shift, vertical flip, random cropping, random erasing, random rotation, random translation, Cutout, CutMix, Adversarial Training, and others can be applied effectively using automation methods (e.g., RandAugment, AutoAugment, AugLy, AutoFlip, DeepAugment, and others) to improve DL generalization capabilities.

Pediatric 12-lead ECGs typically have the following features:
a. Higher heart rate
b. Lower T-wave amplitude relative to QRS amplitude
c. Greater QRS peak-to-peak amplitude
d. More leads with highly biphasic QRS complexes
e. Narrower/shorter QRS duration
f. Shorter PR interval
g. T-wave inversions on leads V1 to V3
h. Slightly peaked P-wave
i. Slightly prolonged QTC
j. Greater amplitude/duration/area of Q-wave in left precordial leads (V5 and V6).

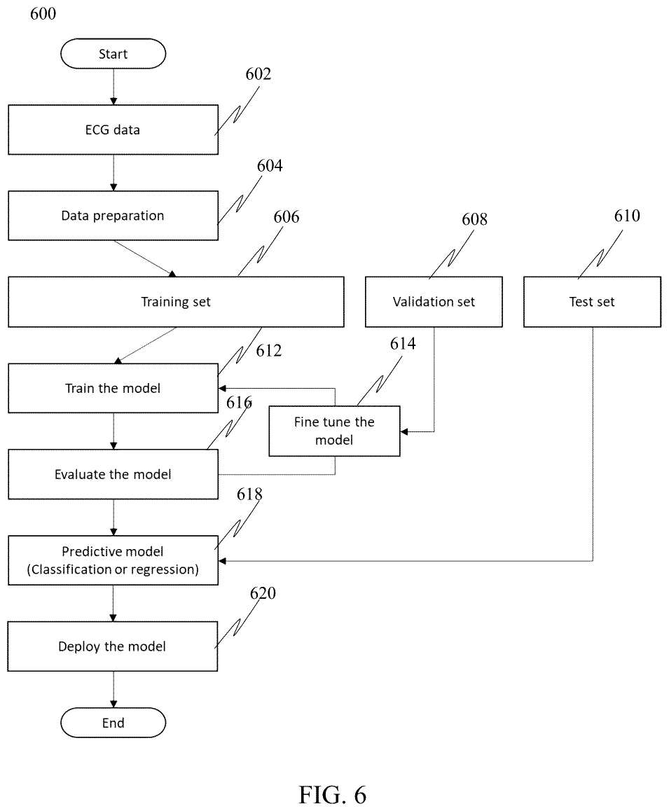

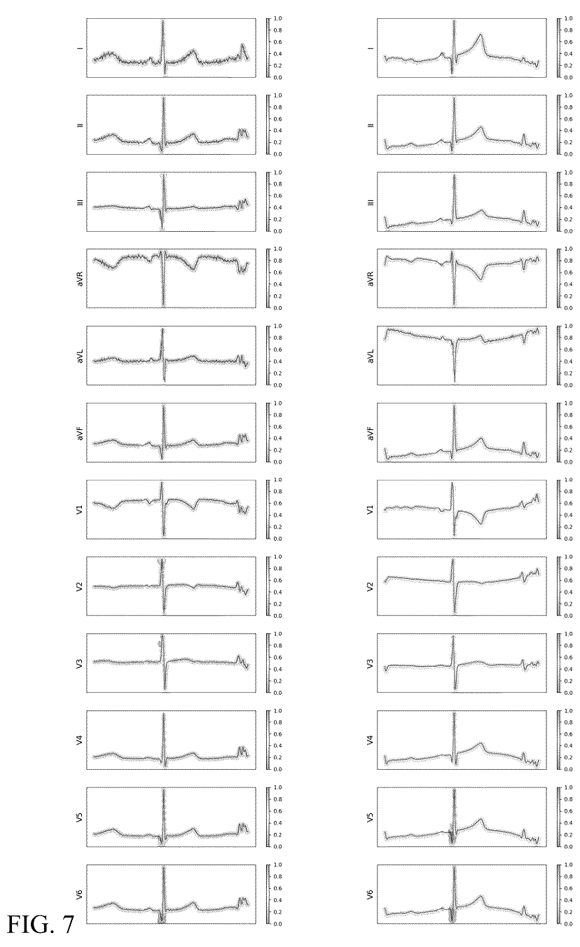

FIG. 7, in accordance with an embodiment, displays the examples of the ECG signal and corresponding interpretations for pediatric cases, using the Deep Learning (DL) model that was trained and tested to determine the significance of various waves in 12-lead ECG for pediatric and age group classification. AI explainability techniques were employed such as SHAP (SHapley Additive explanations) in FIG. 6, and other potential methods may include GradCAM, LIME, and more.

Referring to FIG. 6, that illustrates a flowchart to train, text, and deploy the feature-based ML and end-to-end DL models, in accordance with an embodiment. Flowchart 600 begins at step 602 with ECG data. At step 604 data preparation is performed such that a training set is created at step 606. At step 612 the model is trained. At step 616 the model is evaluated and the model can be fine tuned at step 614 with inputs from validation set 608. Step 618 results in a predictive model (classification or regression) that includes inputs from test set 610. Accordingly, at step 620 the model is deployed.

Referring again to FIG. 2, in one embodiment, is a flowchart of a method 100 for analysis of an ECG input for a subject to determine whether the subject is a pediatric subject or non-pediatric subject, and to classify the subject in one of a plurality of different age groups based on an estimated age of the subject. The methods described in connection with the figures are provided as examples only, and shall be understood not to limit the scope of the disclosure.

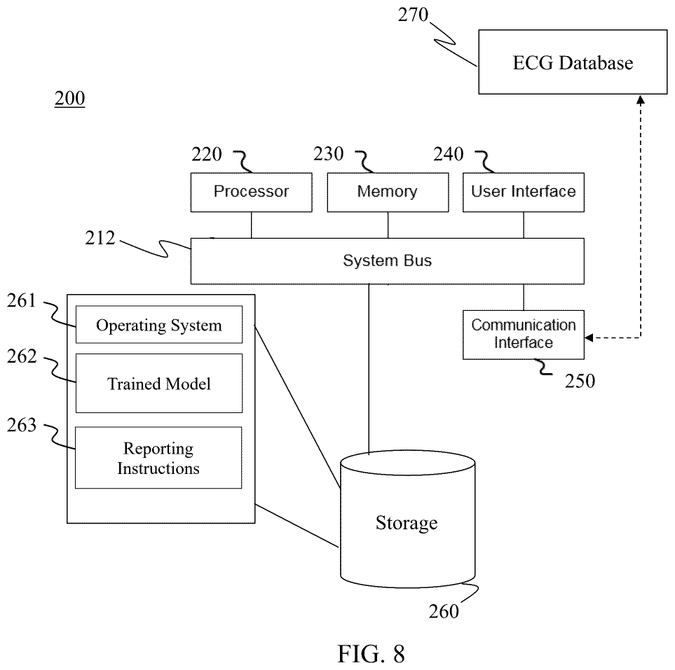

At step 110 of the method, an ECG analysis system 200 is provided. Referring to an embodiment of a diagnostic platform 200 as depicted in FIG. 8, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the ECG analysis system 200 may be different and more complex than illustrated. Additionally, ECG analysis system 200 can be any of the devices described or otherwise envisioned herein. Other elements and components of the ECG analysis system 200 are disclosed and/or envisioned elsewhere herein.

At step 120 of the method, the ECG analysis system 200 receives ECG input for the subject. The ECG input can be any ECG trace or signal obtained from a person, and can be received from any source, local or remote. For example, ECG analysis system 200 optionally comprises an ECG database from which an ECG trace or signal can be obtained. Notably, the ECG input does not comprise or is not accompanied with an age for the subject.

At step 122 of the method, the system extracts one or more features from the received ECG input, wherein the extracted one or more features are provided as input to the trained algorithm. The features can be any of the features described or otherwise envisioned herein, and can be extracted using known methods for extracting features, including for extracting features from ECG data.

At step 130 of the method, a trained algorithm determines, using the received ECG input, that the subject is a pediatric or non-pediatric subject. The trained algorithm makes the determination as described or otherwise envisioned herein. According to one embodiment, the algorithm uses extracted features from the ECG input as input to the algorithm, and makes the determination as output of the algorithm.

According to an embodiment, the trained algorithm is a trained machine learning algorithm or an end-to-end deep learning algorithm. According to an embodiment, the algorithm is trained using multi-lead ECG data for a plurality of subjects at a plurality of different ages. According to an embodiment, a plurality of features are extracted from the multi-lead ECG data for each of the plurality of subjects, and wherein the plurality of extracted features are used to train the algorithm. According to an embodiment, the plurality of extracted features are reduced via dimensionality reduction prior to training the algorithm.

At optional step 124 of the method, the system receives, from a clinician via a user interface, a selection of a preferred age range, wherein the determined one of a plurality of different non-pediatric age groups or determined one of a plurality of different non-pediatric age groups in which the subject belongs comprises the preferred age range.

At step 140 of the method, upon determining that the subject is a pediatric subject, the trained algorithm determines that the subject belongs in one of a plurality of different pediatric age groups based on the received ECG input. Alternatively, at step 150 of the method, upon determining that the subject is a non-pediatric subject, the trained algorithm determines that the subject belongs in one of a plurality of different non-pediatric age groups based on the received ECG input. The plurality of different pediatric age groups and the plurality of different non-pediatric age groups can be any of the age groups described or otherwise envisioned herein. According to one embodiment, the algorithm uses extracted features from the ECG input as input to the algorithm, and makes the age group determination as output of the algorithm.

At step 160 of the method, the system performs an automated analysis of the received ECG input for the subject, wherein the automated analysis is based in part on the one of a plurality of different pediatric age groups or the one of a plurality of different non-pediatric age groups in which the subject belongs. The automated analysis of the received ECG input for the subject can be performed using an automated ECG analysis tool.

At step 170 of the method the system displays, via a user interface, the determined one of a plurality of different non-pediatric age groups or determined one of a plurality of different non-pediatric age groups in which the subject belongs. Optionally, the system displays the results of the automated analysis.

Referring again to FIG. 8 is a schematic representation of an ECG analysis system 200. System 200 may be any of the devices or systems described or otherwise envisioned herein, and may comprise any of the components described or otherwise envisioned herein. It will be understood that FIG. 2 constitutes, in some respects, an abstraction and that the actual organization of the components of system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 may also comprise or be in direct or indirect communication with an ECG database from which an ECG input, such as an ECG trace or signal, can be obtained or received. According to an embodiment, the ECG database may be a local or remote database and is in direct and/or indirect communication with system 200. Thus, according to an embodiment, the system comprises an ECG database.

According to an embodiment, storage 260 of system 200 may store one or more algorithms, modules, and/or instructions to carry out one or more functions or steps of the methods described or otherwise envisioned herein. For example, storage 260 may comprise, among other instructions or data, a trained algorithm 262, and/or reporting instructions 263.

According to an embodiment, the trained algorithm 262 of the system is trained to analyze the received input, including but not limited to the ECG input for a subject, to generate output, including but not limited to a determination of whether the subject is a pediatric subject or non-pediatric subject, and a classification of the subject in one of a plurality of different age groups based on an estimated age of the subject. The trained algorithm 262 can be any model that can be trained to utilize the input to generate the output, as described or otherwise envisioned herein. For example, the model can be a neural network or other trained machine learning model, for example a convolutional neural network (CNN) or a transformer network or other neural network. Thus, according to an embodiment, the system 200 comprises a trained algorithm 262 that receives the input data and outputs the output, as described or otherwise envisioned herein.

According to an embodiment, reporting instructions 265 directs the system to provide the output of the system-such as the determined one of a plurality of different non-pediatric age groups or determined one of a plurality of different non-pediatric age groups in which the subject belongs, and/or the results of the automated analysis—to a patient, clinician, or to another device or system. The provided output can be any of the information as described or otherwise envisioned herein. The system may provide the information to a user via any mechanism, including but not limited to a visual display. The information may be communicated by wired and/or wireless communication to another device. For example, the system may communicate the information to a monitor, screen, mobile phone, computer, laptop, wearable device, and/or any other device configured to allow display and/or other communication of the information.

According to an embodiment, system 200 is configured to process many thousands or millions of datapoints in the input data used to train the algorithm 262. For example, generating a functional and skilled trained algorithm from a corpus of training data requires processing of millions of datapoints from input data and generated features. This can require millions or billions of calculations to generate a novel algorithm from those millions of datapoints and millions or billions of calculations. As a result, each trained algorithm is novel and distinct based on the input data and parameters of the model, and thus improves the functioning of the system. Generating a functional and skilled trained algorithm comprises a process with a volume of calculation and analysis that a human brain cannot accomplish in a lifetime, or multiple lifetimes.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for analysis of an ECG input for a subject to determine whether the subject is a pediatric subject or non-pediatric subject, and to classify the subject in one of a plurality of different age groups based on an estimated age of the subject, comprising:
receiving an ECG input for the subject, wherein the ECG input does not comprise or is not accompanied with an age for the subject;
determining, by a trained algorithm based on the received ECG input, that the subject is a pediatric or non-pediatric subject;
upon determining that the subject is a pediatric subject, determining, by the trained algorithm based on the received ECG input, that the subject belongs in one of a plurality of different pediatric age groups;
upon determining that the subject is a non-pediatric subject, determining by the trained algorithm based on the received ECG input, that the subject belongs in one of a plurality of different non-pediatric age groups;
performing, using an automated ECG analysis tool, an automated analysis of the received ECG input for the subject, wherein the automated analysis is based in part on the one of a plurality of different pediatric age groups or the one of a plurality of different non-pediatric age groups in which the subject belongs.

2. The method of claim 1, further comprising the step of extracting one or more features from the received ECG input, wherein the extracted one or more features are provided as input to the trained algorithm.

3. The method of claim 1, wherein the trained algorithm is a trained machine learning algorithm or an end-to-end deep learning algorithm.

4. The method of claim 3, wherein the algorithm is trained using multi-lead ECG data for a plurality of subjects at a plurality of different ages.

5. The method of claim 4, wherein a plurality of features are extracted from the multi-lead ECG data for each of the plurality of subjects, and wherein the plurality of extracted features are used to train the algorithm.

6. The method of claim 5, wherein the plurality of extracted features are reduced via dimensionality reduction prior to training the algorithm.

7. The method of claim 1, further comprising the step of displaying, via a user interface, the determined one of a plurality of different non-pediatric age groups or determined one of a plurality of different non-pediatric age groups in which the subject belongs.

8. The method of claim 7, further comprising the step of displaying, results of the automated analysis.

9. The method of claim 7, further comprising the step of receiving, from a clinician via a user interface, a selection of a preferred age range, wherein the determined one of a plurality of different non-pediatric age groups or determined one of a plurality of different non-pediatric age groups in which the subject belongs comprises the preferred age range.

10. A system for analysis of an ECG input for a subject to determine whether the subject is a pediatric subject or non-pediatric subject, and to classify the subject in one of a plurality of different age groups based on an estimated age of the subject, comprising:
ECG input for the subject, wherein the ECG input does not comprise or is not accompanied with an age for the subject;
a trained algorithm, wherein the trained algorithm is trained to determine whether the subject is a pediatric subject or non-pediatric subject, and to classify the subject in one of a plurality of different age groups based on an estimated age of the subject;
a processor configured to: (i) determine, by the trained algorithm based on the ECG input, that the subject is a pediatric or non-pediatric subject; (ii) upon determining that the subject is a pediatric subject, determine, by the trained algorithm based on the ECG input, that the subject belongs in one of a plurality of different pediatric age groups or upon determining that the subject is a non-pediatric subject, determine by the trained algorithm based on the ECG input, that the subject belongs in one of a plurality of different non-pediatric age groups; and (iii) perform, using an automated ECG analysis tool, an automated analysis of the received ECG input for the subject, wherein the automated analysis is based in part on the one of a plurality of different pediatric age groups or the one of a plurality of different non-pediatric age groups in which the subject belongs.

11. The system of claim 10, wherein the processor is further configured to extract one or more features from the ECG input, wherein the extracted one or more features are provided as input to the trained algorithm.

12. The system of claim 10, wherein the trained algorithm is a trained machine learning algorithm or an end-to-end deep learning algorithm.

13. The system of claim 10, wherein the processor is further configured to cause the display, via a user interface, of the determined one of a plurality of different non-pediatric age groups or determined one of a plurality of different non-pediatric age groups in which the subject belongs.

14. The system of claim 10, wherein the processor is further configured to cause the display, via a user interface, of results of the automated analysis.

15. The system of claim 10, wherein the processor is further configured to receive, from a clinician via a user interface, a selection of a preferred age range, wherein the determined one of a plurality of different non-pediatric age groups or determined one of a plurality of different non-pediatric age groups in which the subject belongs comprises the preferred age range.
